(19) United States
(12) Patent Application Publication
(10) Pub. No.: US 2010/0040620 A1
(43) Pub. Date: Feb. 18, 2010

(54) ANTI-CANCER ANTIBODIES AGAINST LEWISy AND LEWISb ANTIGENS

(76) Inventors: Linda Gillian Durrant, Nottingham (GB); Philip Anthony Jennings, Warrawee, New South Wales (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/441,637

(22) PCT Filed: Sep. 20, 2007
(86) PCT No.: PCT/AU2007/001386

§ 371 Date: Jun. 19, 2009

(30) Foreign Application Priority Data
Sep. 20, 2006 (AU) 2006905191

(51) Int. Cl. A61K 39/395; C07K 16/00; C12N 5/02

(52) U.S. Cl. 424/138.1; 530/387.1; 435/375

(57) ABSTRACT

The present invention provides a binding complex comprising antibodies or antigen-binding fragments which bind to both Lewis^{y} and Lewis^{b} antigens, wherein the antibodies or antigen-binding fragments are in the form of multimers, and wherein the antibodies or antigen-binding fragments do not naturally form multimers.

### FIELD OF THE INVENTION

The present invention relates to a binding complex comprising antibodies or antigen binding fragments useful for the treatment of cancers. In particular the present invention provides a binding complex comprising antibodies or antigen-binding fragments which bind to both Lewis^{y} and Lewis^{b} antigens, wherein the antibodies or antigen-binding fragments are in the form of multimers. The binding complex induces cell death in tumour cells over-expressing Lewis^{y} and Lewis^{b}.

### BACKGROUND OF THE INVENTION

Lewis^{y} and Lewis^{b} are complex carbohydrates over-expressed by breast, lung, colon and ovarian cancers. They may therefore represent good targets for monoclonal antibody (mAb) therapy. The Lewis^{y} hapten is a difucosylated tetrasaccharide (Fucα1-2Galβ1-4(Fucα1-3)GlcNAc) found on type 2 blood group oligosaccharides. This antigen is a positional isomer of the Lewis^{b} hapten (Fucα1-2Galβ1-3(Fucα1-4)GlNAc and a fucosylated derivative of the Lewis^{x} hapten (Abe et al. (1986) Cancer Research 46:2639; Kim et al. (1986) Cancer Research 46:5985). Lewis^{y} is expressed on breast, bronchus, pancreas, the genitourinary system and within deep glands of the gastrointestinal tract. In contrast Lewis^{b} is expressed by the surface epithelium (Sakamoto et al. (1989) Cancer Research 49:745-52; Kitamura et al. (1994) Proc. Natl. Acad. Sci. 91:12957-61).

The IgM mouse mAb, C14 was raised against primary colorectal tumour cells using standard fusion protocols and binds to both Lewis^{y} and Lewis^{b} (extended and non-extended) antigens (Brown et al. (1983) Biosci. Rep. 3:163; Brown et al. (1984) Int. J. Cancer 33:727; Durrant et al. (1993) Hybridoma 12:647-60). The C14 antibody bound to 78% of colorectal cancers (Durrant et al. (1989) J. Natl. Cancer Inst. 81:688-95) but as a murine IgM it was unsuitable for in vivo studies. To produce an IgG variant of the antibody, rats were immunised with C14 mAb and purified rat anti-C14 produced. Immunisation of mice with this antiserum and C14gp200 antigen, followed by the fusion of their splenocytes with a mouse myeloma resulted in the production of five IgG mAbs, two IgG3s (SC101/23, SC101/29 mAb) and three IgG1s (SC101/33, SC101/42 and SC101/43; the five having previously been published as the “692” mAbs). All the IgG variants recognised the Lewis^{y} and Lewis^{b} antigens and demonstrated the same specificity as C14. Further, these antibodies were shown by thin layer chromatography and ELISA to bind to extended and non-extended Lewis^{y} and Lewis^{b} haptens but not to Lewis^{x} or H blood group haptens (Brown et al (1983) Biosci. Rep. 3:163).

Antibodies which bind to both Lewis^{y} and Lewis^{b} antigens are known, however, SC101 mAbs are unique in their ability to recognise both Lewis^{y} and Lewis^{b} determinants. No other mAb and only one rare lectin recognise a similar facet of these two molecules. Lewis y/b is predominantly expressed on a ceramide backbone as a glycolipid. Recent crystallographic studies have shown that antibodies specific to Lewis^{y} can have very different binding sites which accommodate either the N-acetlyl-glucosamine or the fucose residues (Ramsland et al. (2004) J. Mol. Biol. 340:809-18) within the hapten. SC101 is different again as its binding site accommodates an aspect of both Lewis^{y} and Lewis^{b} which is very unusual as they are stereo isomers of each other. Of further interest is that these antibodies cannot recognise the glycolipid in most normal tissues including the gastrointestinal tract probably due to steric hindrance from the ceramide or other closely associated molecules. This gives the SC101 antibodies a unique tissue distribution but very strong binding to a range of epithelial tumours (Brown et al. (1983) Biosci. Rep. 3:163).

During initial characterisation of binding of these antibodies to primary disaggregated colorectal tumours, it was observed that they induced cell death. Cell death can result via a number of mechanisms including apoptosis and oncosis. Apoptosis is caspase dependent and is marked by cellular shrinking, condensation and margination of chromatin ruffling of the plasma membrane with eventual breaking up of the cell into apoptotic bodies. Oncosis is early stage necrotic cell death and is marked by cellular swelling as a result of progressive membrane permeability phases. Necrosis refers to the morphological alterations that appear after cell death and can occur following apoptosis or oncosis.

The present inventors have now demonstrated tumour cell killing by SC101/29 mAb which antibody directly kills tumour cells over-expressing Lewis^{y} and Lewis^{b} both in vitro and in vivo by a unique mechanism that has a number of similarities to classical oncosis.

### SUMMARY OF THE INVENTION

In a first aspect of the present invention there is provided a binding complex comprising antibodies or antigen-binding fragments which bind to both Lewis^{y} and Lewis^{b} antigens, wherein the antibodies or antigen-binding fragments are in the form of multimers.

In a second aspect of the present invention there is provided for inducing cell death in tumour cells over-expressing Lewis^{y} and Lewis^{b} antigens, comprising administering to a subject an effective amount of a binding complex according to the first aspect of the invention.

In a third aspect of the present invention there is provided a method for treating cancer in a subject in need thereof comprising administering to the subject a therapeutically effective amount of a binding complex according to the first aspect of the invention.

In a fourth aspect of the present invention there is provided a pharmaceutical composition for inducing cell death in tumour cells over-expressing Lewis^{y} and Lewis^{b} antigens comprising a binding complex according to the first aspect of the invention together with a pharmaceutically acceptable carrier or diluent.

### BRIEF DESCRIPTION OF THE FIGURES

FIG. 1a shows binding of SC101 to freshly disaggregated colorectal tumour cells, as assayed by indirect immunofluorescence and analysed by flow cytometry. Each bar refers to the mean fluorescence for an individual tumour. The binding of SC101 to the colorectal tumour cell lines, Colo205, C170, HT29 and LoVo are shown for comparison.

FIG. 1b shows IC50 growth inhibition curves of LoVo and C170 cells by SC101/29 mAb. The % inhibition of growth expressed as the number of cells exposed to the SC101/29 mAb/number of cells exposed to control mAb is shown. The number of viable cells from the growth inhibition curves were determined by MTS taking optical density readings at 490 nm.

FIG. 1c shows IC50 growth inhibition curves of Colo205 and HT-29 cells by SC101/29 mAb. The % inhibition of growth expressed as the number of cells exposed to the SC101/29 mAb/number of cells exposed to control mAb is shown. The number of viable cells from the growth inhibition curves were determined by MTS taking optical density readings at 490 nm.

FIG. 2a shows 2×10⁵ Colo205 cells were treated for 4 hr with either 30 μg/ml IgG, 300 ng/ml anti-Fas clone Ch11, 30 μg/ml BR96 or 30 μg/ml SC101/29 mAb for 30 min, 2 hr or 4 hr. Cells were stained with PI for 15 mins at room temperature in the dark and analysed with a FC500 flow cytometer 488 nm (emission 620 nm FL3).

FIG. 2b shows 2×10⁵ C170 cells were treated for 4 hr with either 30 μg/ml IgG, 300 ng/ml anti-Fas clone Ch11, 30 μg/ml BR96 or 30 μg/ml SC101/29 mAb for 30 min, 2 hr or 4 hr. Cells were stained with PI for 15 mins at room temperature in the dark and analysed with a FC500 flow cytometer 488 nm (emission 620 nm FL3).

FIG. 2c shows 2×10⁵ HT-29 cells were treated for 4 hr with either 30 μg/ml IgG, 300 ng/ml anti-Fas clone Ch11, 30 μg/ml BR96 or 30 μg/ml SC101/29 mAb for 30 min, 2 hr or 4 hr. Cells were stained with PI for 15 mins at room temperature in the dark and analysed with a FC500 flow cytometer 488 nm (emission 620 nm FL3).

FIG. 2d shows 2×10⁵ LoVo cells were treated for 4 hr with either 30 μg/ml IgG, 300 ng/ml anti-Fas clone Ch11, 30 μg/ml BR96 or 30 μg/ml SC101/29 mAb for 30 min, 2 hr or 4 hr. Cells were stained with PI for 15 mins at room temperature in the dark and analysed with a FC500 flow cytometer 488 nm (emission 620 nm FL3).

FIG. 2e shows 2×10⁵ Colo205, C170, HT-29 or LoVo cells were treated for 4 hr with 1-100 μg/ml SC101/29 mAb for 30 min in the presence of 0-10 μM doxorubicin. Cells were analysed with a FC500 flow cytometer 488 nm (emission 575 nm FL2).

FIG. 2f shows cells were stained with a mAb recognising p-glycoprotein (0.3-30 μg/ml) and analysed with a FC500 flow cytometer 488 nm (emission 525 FL1).

FIG. 3a shows 2×10⁵ C170, cells were treated for 1 hr with either SC101/29, SC101/43 or both mAbs. Cells were stained with PI for 15 mins at room temperature in the dark and analysed with a FC500 flow cytometer 488 nm (emission 620 nm FL3).

FIG. 3b shows 2×10⁵ C170, cells were treated for 1 hr with either 100 μg/ml IgG, 100 μg/ml IgG mixed with avidin, 100 μg/ml SC101/29, 100 μg/ml SC101/43-biotin or 100 μg/ml of biotinylated SC101/43 cross-linked with avidin. Cells were stained with PI for 15 mins at room temperature in the dark and analysed with a FC500 flow cytometer 488 nm (emission 620 nm FL3).

FIG. 3c shows 1×10⁵ Colo205 cells were treated for 30 min with 100 μg/ml mouse SC101/29-human IgG₂ chimeric antibody followed by cross-linking anti-human IgG antibody (0-100 μg/ml). After 3 h incubation at room temperature cells were stained with 7AAD for 20 min in the dark and analysed with a Cell Quanta SC MPL 488 nm. The mean percentage of dead (7AAD⁺) cells in two samples is shown.

FIG. 3d shows 5×10 C170 cells were treated for approximately 12 hrs with 100 μg/ml isotype negative control mAb, 100 ng/ml anti-Fas or 100 μg/ml SC101/29 mAb in the presence of either 0, 1 or 10 μM z-FMK-vad pan-caspase inhibitor. Cells were stained with annexin V and PI for 15 min in the dark at room temperature and analysed using a FC500 flow cytometer 488 nm (emission 525 nm FL1, 620 nm FL3).

FIG. 3e shows 2×10⁶ C170 cells were exposed to IgG (30 μg/ml) negative control for 4 hr, SC101 1-4 hr, or 0.5M sorbitol 30 min (positive control). Cells were extracted in ice cold Tris extraction buffer+0.5 mM NaVO4, and 75 μg total protein loaded per lane on SDS-PAGE. 10% gels were separated at 130V for 90 min and transferred on a BioRad semi-dry blotter (12V constant voltage) for 70 min onto PVDF. The membranes were blocked and probed with 1μl per 2 ml P-p38, P-JNK or P-ERK (1% BSA TBS-T, washed and developed with ECL Plus+.

FIG. 4a shows a graph demonstrating the effect of SC101/29 mAb, 5-FU/leucovorin or a combination of SC101/29 mAb and 5-FU/leucovorin on the growth of C170 xenografts growing in nude mice. Growth of C170 xenografts was measured at days 12, 16, 19 and 23 by measurement of cross-sectional area (mm²) when animals were treated with either SC101/29 mAb ip (0.2 mg), control antibody ip (0.2 mg) and 5-FU/leucovorin (12.5 mg/kg; iv). Analysis of variance of the results from day 23 showed the significant values of p<0.004 when comparing SC101/29 mAb to the untreated control group and p<0.020 for the control group plus 5-FU/leucovorin to SC101/29 mAb plus 5-FU/leucovorin.

FIG. 4b shows termination tumour weights of mice not treated (group 1), immunised with SC101/29 mAb (group 2) alone, 5-FU/leucovorin alone (group 3) or the combination of SC101/29 mAb and 5-FU/leucovorin (group 4).

FIG. 4c shows weight of mice not treated, immunised with SC101/29 mAb alone, 5-FU/leucovorin alone or the combination of SC101/29 mAb and 5-FU/leucovorin.

FIG. 4d shows survival data of animals with C170 xenografts treated with SC101/29 mAb ip (0.2 mg), 5-FU/leucovorin (12.5 mg/kg iv) or SC101/29 mAb (0.2 mg) and 5-FU/leucovorin (12.5 mg/kg iv) in combination. SC101/29 mAb was given on day 7 then 3 times weekly. 5-FU/leucovorin was administered on days 1, 3, 5 and 7. SC101/29 mAb when administered in combination with 5-FU/leucovorin significantly enhanced survival over the vehicle control mice (p=0.0163 Log Rank).

FIG. 5 shows a graph representing the effect of 10 μg or 100 μg SC101/29 mAb, or the vehicle control, on the final liver tumour weights (g) in the C170HM2 liver metastases nude mouse model.

### DETAILED DESCRIPTION OF THE INVENTION

The present inventors have demonstrated that a binding complex comprising antibodies or antigen-binding fragments which bind both Lewis^{y} and Lewis^{b} antigens induce cell death in tumour cells over-expressing Lewis^{y} and Lewis^{b}.

In a first aspect of the present invention there is provided a binding complex comprising antibodies or antigen-binding fragments which bind to both Lewis^{y} and Lewis^{b} antigens, wherein the antibodies or antigen-binding fragments are in the form of multimers.

The term “antibody” as used herein, is intended to refer to immunoglobulin molecules comprised of four polypeptide chains, two heavy (H) chains and two light (L) chains inter-connected by disulfide bonds. Each heavy chain is comprised of a heavy chain variable region (HCVR or V_{H}) and a heavy chain constant region. The heavy chain constant region comprises three domains, C_{H}1, C_{H}2 and C_{H}3. Each light chain is comprised of a light chain variable region (LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

The term “antigen-binding fragment” of an antibody, as used herein refers to one or more components or derivatives of an immunoglobulin that exhibit the ability to bind to an antigen. It has been shown that the antigen-binding function of an antibody can be performed by fragments of a fall length antibody. Examples of binding fragments encompassed within the term “antigen-binding fragment” of an antibody include (i) a Fab fragment, a monovalent fragment consisting of the V_{L}, Vₕ, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al (1989) Nature 341:544-546) which consists of a single V_{H} domain, or a V_{L} domain (van den Beuken et al. (2001) J. Mol. Biol, 310, 591); (vi) an isolated complementarity determining region (CDR); and (vii) complementarity determining regions fused through a cognate framework region, such as those described in Qiu et al. (2007) Nature Biotechnology 25(8):921-929. Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); (Bird et al. (1988) Science 242:423-426; Huston et al. (1988) Proc. Natl. Acad. Sci. USA 85:5879-5883). Such single chain Fvs are also intended to be encompassed within the term “antigen-binding fragment” of an antibody. Other forms of single chain Fvs and related molecules such as diabodies or triabodies are also encompassed. Diabodies are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g. Holliger et al. (1993) Proc. Natl. Acad. Sci. USA 90:6444-6448; Poljak et al. (1994) Structure 2:1121-1123).

It is a necessary feature of the present invention that the antibodies or antigen-binding fragments are in the form of multimers. The antibodies or antigen-binding fragments bind both Lewis^{y} and Lewis^{b} antigens causing the antigens to become cross-linked. The cross-linking of Lewis^{y} and Lewis^{b} antigens may be important in the mediation of tumour cell death for cells which over-express Lewis^{y} and Lewis^{b}.

The term “multimers” as used herein is meant two or more antibodies or antigen-binding fragments which form an association with each other. Furthermore, the multimer may be a homo- or hetero-multimer. For example, the multimer may be a homo- or hetero-dimer, homo- or hetero-trimer or higher. Homo-multimers are multimers which comprise the identical antibodies or antigen-binding fragments, while hetero-multimers are multimers which comprise at least two dissimilar antibodies or antigen-binding fragments. However, regardless of the multimer composition each antibody or antigen-binding fragment must bind to both Lewis^{y} and Lewis^{b} antigens.

In a preferred embodiment of the present invention the multimer is a homo- or hetero-dimer.

In yet a further preferred embodiment of the present invention the antibodies do not naturally dimerise or multimerise.

The term “binds both Lewis^{y} and Lewis^{b} antigens” as used herein is meant an antibody or antigen-binding fragment which binds to both Lewis^{y} and Lewis^{b} antigens such that the Lewis^{y} and Lewis^{b} antigens become cross-linked.

The term “binds to” as used herein, is intended to refer to the binding of an antigen by an immunoglobulin variable region of an antibody with a dissociation constant (Kd) of 1 μM or lower as measured by surface plasmon resonance analysis using, for example a BIAcore™ surface plasmon resonance system and BIAcore™ kinetic evaluation software (eg. version 2.1). The affinity or dissociation constant (Kd) for a specific binding interaction is preferably about 500 nM to about 50 pM, more preferably about 500 nM or lower, more preferably about 300 nM or lower and preferably at least about 300 nM to about 50 pM, about 200 nM to about 50 pM, and more preferably at least about 100 nM to about 50 pM, about 75 nM to about 50 pM, about 10 nM to about 50 pM.

In certain embodiments of the present invention the antibody or antigen-binding fragment is a diabody or multibody. In other embodiments of the present invention the diabody or multibody comprises a modified Fc domain.

In other embodiments of the present invention the multimer comprising antibodies or antigen-binding fragments is an IgG antibody. In a further embodiment of the present invention the multimer is a dimeric IgG1 antibody.

While some antibodies (e.g. IgG3) form dimers in vitro and in vivo, IgG1 antibodies exist in a monomeric form. In order that monomeric antibodies or antigen-binding fragments thereof may induce tumour cell death in tumour cells over-expressing Lewis^{y} and Lewis^{b} antigens, the Lewis^{y} and Lewis^{b} antigens are cross-linked by avidin/biotin cross-linking of monomeric antibodies or antigen-binding fragments. Alternatively, the monomeric antibodies or antigen-binding fragments may be cross-linked by recombinant engineering, for example, through mutation of a serine to cysteine in the C_{H}3 region gene, allowing interchain disulfide bond formation at the carboxy terminal of the monomer (Caron et al. (1992) J. Exp. Med. 1191-1195). This in turn facilitates cross-linking of Lewis^{y} and Lewis^{b} antigens.

In a second aspect of the present invention there is provided a method for inducing cell death in tumour cells over-expressing Lewis^{y} and Lewis^{b} antigens, comprising administering to a subject an effective amount of a binding complex comprising antibodies or antigen-binding fragments which bind to both Lewis^{y} and Lewis^{b} antigens, wherein the antibodies or antigen-binding fragments are in the form of multimers.

In a third aspect of the present invention there is provided a method for treating cancer in a subject in need thereof comprising administering to the subject a therapeutically effective amount of a binding complex according to the first aspect of the invention, which binding complex binds to both Lewis^{y} and Lewis^{b} antigens.

The term “therapeutically effective amount” refers to an amount of an antibody or antigen-binding fragment thereof (including pharmaceutical compositions comprising the antibody or antigen-binding fragment thereof) sufficient to treat or ameliorate a specified disease or disorder or one or more of its symptoms and/or to prevent or reduce the occurrence of the disease or disorder, in the case of the present invention cancer.

When used with respect to methods of treatment and the use of the antibody or antigen-binding fragment thereof (including pharmaceutical compositions comprising the antibody or antigen-binding fragment thereof), an individual “in need thereof” may be an individual who has been diagnosed with or previously treated for cancer.

In a preferred embodiment of the present invention cancer is selected from the group consisting of breast, lung, colon and ovarian cancer.

In a fourth aspect of the present invention there is provided a pharmaceutical composition comprising a binding complex according to the first aspect of the invention together with a pharmaceutically acceptable carrier or diluent for inducing cell death in tumour cells over-expressing Lewis^{y} and Lewis^{b} antigens.

A “pharmaceutically acceptable carrier or diluent” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, and the like as well as combinations thereof. In many cases it will be preferable to include isotonic agents, for example, sugars, polyalcohols such as mannitol, sorbitol, or sodium chloride in the composition.

The composition may be in a variety of forms, including liquid, semi-solid or solid dosage forms, such as liquid solutions (e.g. injectable and infusible solutions), dispersions or suspensions, tablets, pills, powders, liposomes or suppositories. Preferably, the composition is in the form of an injectable solution for immunization. The administration may be intravenous, subcutaneous, intraperitoneal, intramuscular, transdermal, intrathecal, and intra-arterial. Preferably the dosage form is in the range of from about 0.001 mg to about 10 mg/kg body weight administered daily, weekly, bi- or tri-weekly or monthly, more preferably about 0.05 to about 5 mg/kg body weight weekly.

The composition may also be formulated as a sterile powder for the preparation of sterile injectable solutions.

In certain embodiments, the binding complex may be prepared with a carrier that will protect the compound against rapid release, such as a controlled release formulation, including implants, transdermal patches, and microencapsulated delivery systems. Compatible polymers may be used such as ethylene vinyl acetate, polyanhydrides, polyglycolic acid, collagen, polyorthoesters or polylactic acid.

The composition may also be formulated for oral administration. In this embodiment, the antibody may be enclosed in a hard or soft shell gelatin capsule, compressed into tablets, or incorporated directly into the subject's diet.

The composition may also be formulated for rectal administration.

The binding complex of the present invention may be administered in order to bind to and identify selected cells in vitro and in vivo, to bind to and destroy selected cells in vivo, or in order to penetrate into and destroy selected cells in vivo.

In the preferred embodiment, the composition is administered to a human.

In order that the nature of the present invention may be more clearly understood, preferred forms thereof will now be described with reference to the following non-limiting examples.

### EXAMPLE 1

Materials and Methods

Cell Lines

C170 is a colorectal cell line derived from primary tumours (Durrant et al. (1986) Br. J. Cancer 53:37-45). Colo205, HT-29, and LoVo are colorectal cell lines obtained from ATCC. All cells were cultured in 10% foetal calf serum (FCS F6178; Sigma, Poole, UK) in RPMI 1640 medium (BE12-702F; Cambrex Bio Science, Berkshire, UK).

Monoclonal Antibodies

SC101 mAb recognising both Lewis^{y} and Lewis^{b} haptens was purified as described previously (Brown et al. (1983) Biosci. Rep. 3:163; Durrant et al. (1993) Hybridoma 12:647-60). The anti-Fas (human activating) clone Ch11 (05-201) antibody was obtained from Upstate and the Br96 and IgG negative control from Sigma (I5381).

Antibody Binding by Colorectal Tumour Cells

Tumour specimens were obtained at the time of colorectal cancer resection. Specimens were finely minced and disaggregated with 0.05% collagenase (Type IV, Boehringer Mannheim, Lewes, UK) for 20 min at 37° C. and stained by indirect immunofluorescence with SC101/29 and goat anti-mouse FITC (Dako Ltd, Bucks UK; 1:100 dilution). Tumour cell lines were aliquoted (10⁵) and stained with SC101/29 mAb or anti-P-glycoprotein (indicating multi-drug resistance, BD Pharmingen, San Diego, Calif. USA) at varying concentrations at 4° C. for 30 min. After washing twice in media (RPMI/10% FCS) cells were incubated with a secondary antibody (goat anti-mouse FITC) at 4° C. for 30 min, before a final wash and analysis on a FC500 flow cytometer 488 nm (emission 525 nm FL1).

In Vitro Killing of Colorectal Tumour Cells

1×10³ colorectal C170, Colo205, HT-29 and LoVo cells were aliquoted into individual wells of a flat bottomed 96-well plate and left to adhere overnight at 37° C. The following day the cells were treated with: 100, 30, 10, 3, 1 or 0 μg/ml of SC101/29 mAb (100 μl/well). An isotype matched positive or negative control antibody was used for comparison. Triplicate wells were used. Cells were left for 5 days in the presence of the antibody at 37° C. prior to the addition of Cell Titer96 Aqueous One Solution (G3580; Promega, Southampton, UK) to each well and the optical density read at 490 nm.

PI uptake

2×10⁵ Colo205, C170, HT-29 or LoVo cells were treated for 4 hr with either 30 μg/ml IgG, 300 ng/ml anti-Fas, 30 μg/ml BR96 or SC101/29 mAbs for 30 min, 2 hrs or 4 hrs. The cells were subsequently stained with PI (630110; BD Biosciences, Oxford, UK) for 15 min in the dark at room temperature prior to being analysed using a FC500 flow cytometer 488 nm (emission 620 nm FL3).

7AAD Uptake

1×10⁵ Colo205 cells were incubated with 100 μg/ml mouse SC101/29-human IgG₂ chimeric antibody at room temperature in phosphate buffered saline (PBS). After 30 min different concentrations of cross-linking anti-human IgG antibody (0-100 μg/ml; Sigma) was added and cells were incubated for 3 hrs at room temperature cells; then cells were stained with 7AAD (Beckman Coulter) for 20 min in the dark and analysed with a Cell Quanta SC MPL 488 nm (Beckman Coulter; emission filter 670LP FL3).

Drug Uptake in MDR Cell Lines

2×10⁵ Colo205, C170, HT-29 or LoVo cells were treated at 37° C. for 30 min with 0, 1, 3, 10, 30 or 100 μg/ml SC101/29 mAb. The cells then had doxorubicin added to a final concentration of 0, 1 or 10 μM and were incubated at 37° C. for 1 h. Cells were washed 5× ice cold phosphate buffered saline (PBS) and analysed for fluorescence using a FC500 flow cytometer 488 nm (emission 575 nm FL2).

Inhibition of Caspase Dependent Cell Death Assay

5×10⁵ C170 cells were aliquoted into individual wells of a 24 well flat bottomed sterile plate and allowed to adhere for 6 hrs at 37° C. The cells were treated with either 100 μg/ml isotype matched negative control mAb, 100 ng/ml anti-Fas or 100 μg/ml SC101/29 mAb and 0, 1 or 10 μM z-FMK-vad (G7232; Perbio Science, UK). The cells were placed back at 37° C. overnight and subsequently stained with annexin V and PI for 15 min and analysed using an FC500 flow cytometer 488 nm (emission 525 nm FL1, 620 nm FL3).

Signalling Via Stress Related Kinases

2×10⁶ C170 cells were exposed to either IgG (30 μg/ml) negative control for 4 hr, SC101 (30 μg/ml) 1-4 hr, or 0.5M sorbitol for 30 min (positive control). Cells were extracted in ice cold extraction buffer (500 mM Tris pH 7.4, 2 mM EDTA, 2 mM Na₄P₂O₇, 2 mM benzamadine, 1 mM PMSF, 0.5 mM NaVO₄, 0.5 mM DTT 0.1% Triton-X-100) the protein concentration measured using a standard BioRad protein assay and 75 μg total protein loaded per lane on SDS-PAGE. 10% gels were separated at 130V for 90 min and transferred on a BioRad semi-dry blotter (12V constant voltage) for 70 min onto PVDF. The membranes were blocked for 1 hr in 1% bovine serum albumin (BSA) Tris buffered saline 0.05% Tween-20 (TBS-T) and probed with 1 μl per 2 ml P-p38, P-JNK or P-ERK (Promega) in 1% BSA TBS-T. The membranes were subsequently washed three times in TBS-T and probed with goat anti-rabbit HRP (GαR HRP; Dako, P0448), washed ×3 in TBS-T and developed with ECL Plus+.

In Vivo Studies

Prevention model. The colorectal tumour cell line, C170 was maintained in serial passage in nude mice. For therapy the mice were sacrificed and the tumours excised. The tumour was finely minced and 3 mm² pieces were implanted, under anaesthetic (Hypnorm, Roche/Hypnovel, Jannsen), subcutaneously, into 40 male mice which had been randomly allocated to 4 experimental groups. A group of mice were treated with 5-FU/leucovorin (12.5 mg/kg) by intravenous (iv) injection on days 1, 3, 5, 7, 21 and 22. Three times weekly mice were also injected intraperitoneally (ip) with 0.2 mg of SC101/29 mAb. Control mice received either SC101/29 mAb, 5-FU/leucovorin or vehicle alone. Tumour size was measured by callipers and tumour cross-sectional area calculated on days 12, 16, 19 and 23. At the termination of the experiment tumours were weighed to assess anti-tumour efficacy. Animals were weighed to assess the toxicity of treatment.

Therapeutic model. The colorectal tumour cell line, C170 was maintained in serial passage in nude mice. For therapy the mice were sacrificed and the tumours excised. The tumour was finely minced and 3 mm3 pieces were implanted, under anaesthetic, subcutaneously into 40 male mice which had been randomly allocated to 4 experimental groups. Mice were explanted with 3 mm³ pieces of C170 xenografts. Groups of mice were treated with 5-FU/leucovorin (25 mg/kg) by iv injection on days 1, 3, 5, 7 and cycled, where applicable, from day 28. Three times weekly mice were also injected iv with SC101/29 mAb (0.2 mg), control mice receiving either SC101/29 mAb alone or control mouse IgG antibody with 5-FU/leucovorin.

Metastases model. The C170HM2 cells were maintained in vitro in RPMI 1640 culture medium containing 10% heat inactivated FCS at 37° C. in 5% CO₂, humidified conditions. Cells from sub-confluent monolayers were harvested with 0.025% EDTA, washed twice in the culture medium outlined above, and re-suspended, for in vivo administration, in sterile PBS, pH 7.4. 1.5×10⁶ cells in a volume of 1 ml were injected into the peritoneal cavity of 30 male nude mice. Animals were allocated to their treatment groups and treatment began on day 1 and continued throughout the study. The groups of mice were treated with either 10 μg or 100 μg of SC101/29 mAb or the vehicle control by iv injection on day 1 and then 3 times weekly. Mice were terminated on day 40 and body and tumour weight evaluated.

Statistics

Statistical analysis was performed using Analysis of Variance and Log Rank on the Minitab programme for the PC.

### EXAMPLE 2

Results

Antibody Binding by Colorectal Tumour Cells

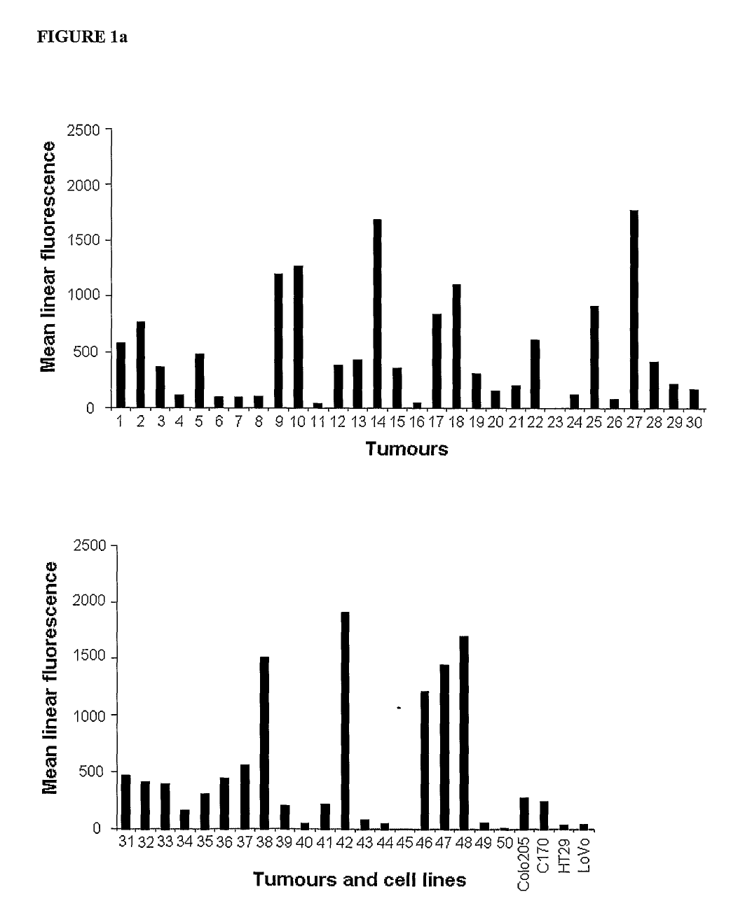

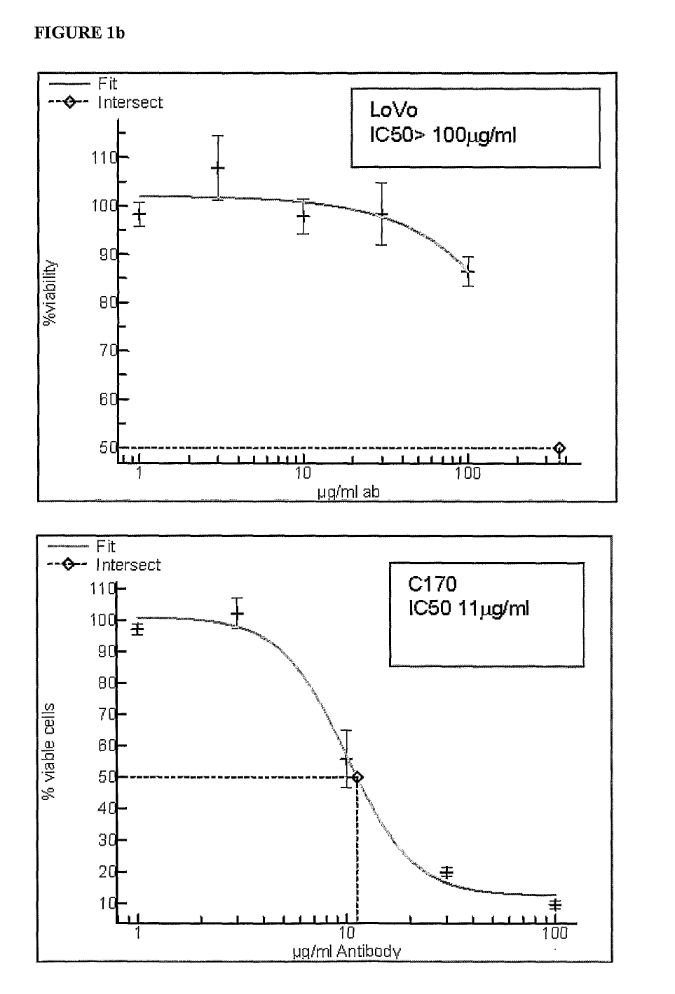

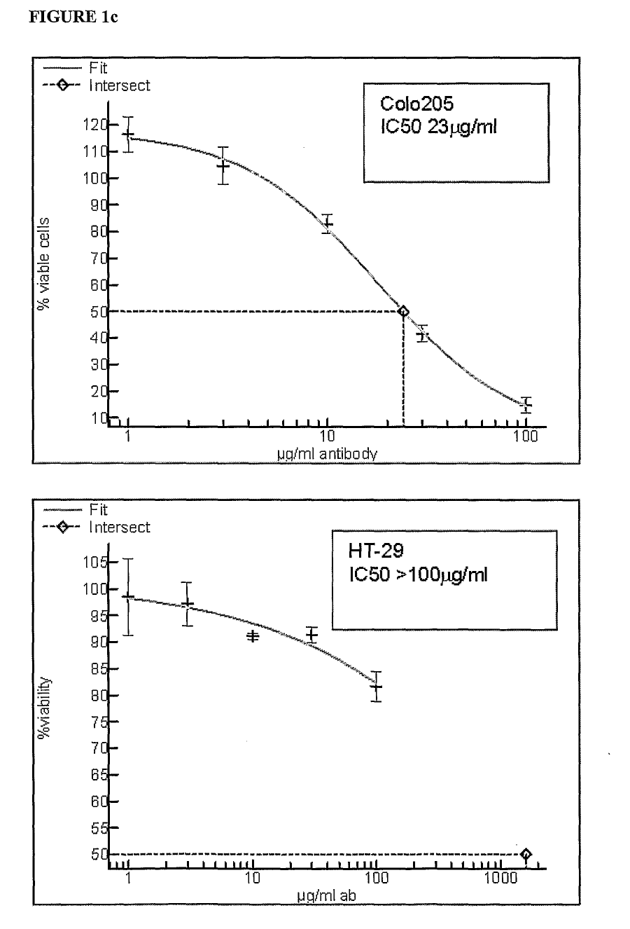

The SC101 mAbs have previously been shown to bind both Lewis^{y} and Lewis^{b} haptens. Strong staining of freshly disaggregated colorectal tumours is shown in FIG. 1a. The mAbs also binds to disaggregated ovarian and gastric tumours (Brown et al. (1983) Biosci. Rep. 3:163). Furthermore, these antibodies induced accelerated cell death of the primary tumour cells. Immunohistochemistry and flow cytometry experiments using both SC101/29 and SC101/33 antibodies has markedly bound to tumour cell lines and tumour xenografts of the following types: ovary, breast, lung, prostate and pancreas (data not shown).

In Vitro Killing of Colorectal Tumour Cells

To investigate this killing further a range of cell lines were screened for binding (FIG. 1a) and inhibition of cell proliferation by the SC101/29 mAb (FIGS. 1b and 1c). C170 and Colo205 cell lines both bound to SC101/29 mAb however the majority of the primary tumours showed stronger binding. In contrast, SC101/29 mAb bound weakly to HT-29 and LoVo at levels equivalent to 20% of the primary tumours. Of particular interest was that SC101/29 mAb inhibited proliferation of C170 and Colo205 cells but failed to inhibit proliferation of HT-29 and LoVo cells that express low levels of the Lewis^{y} and Lewis^{b} antigens.

Viability Test

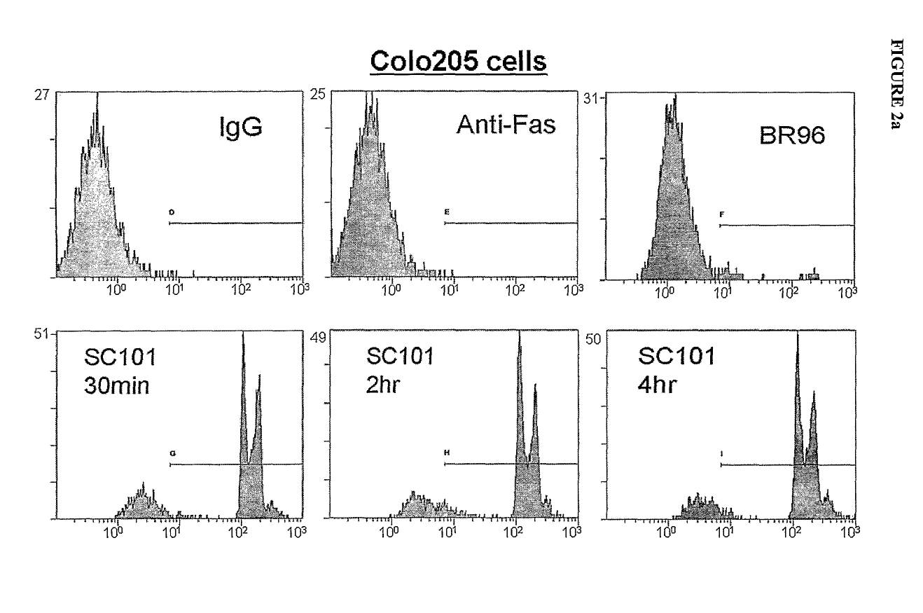

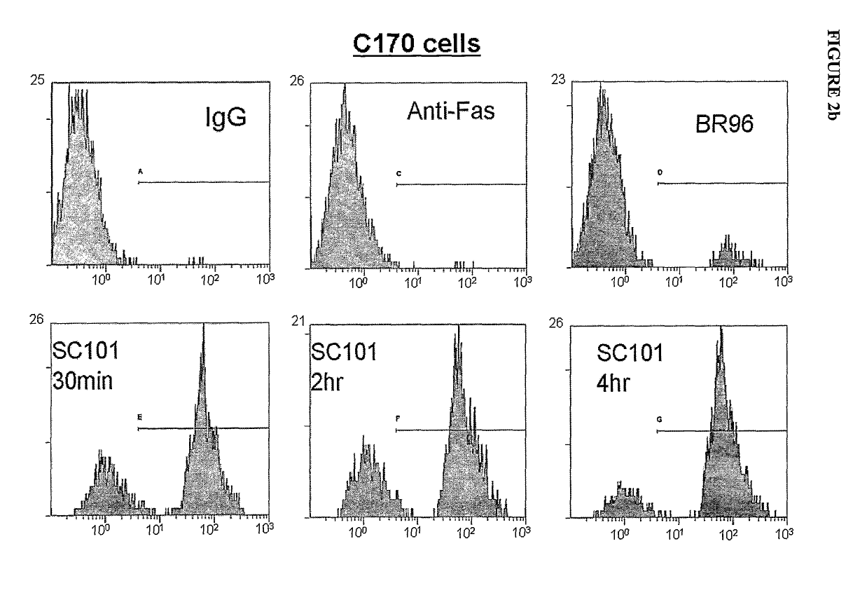

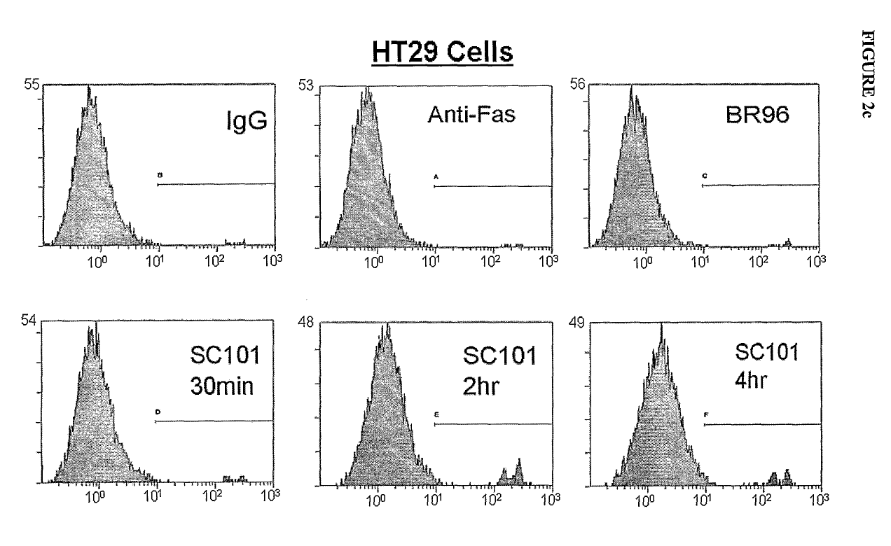

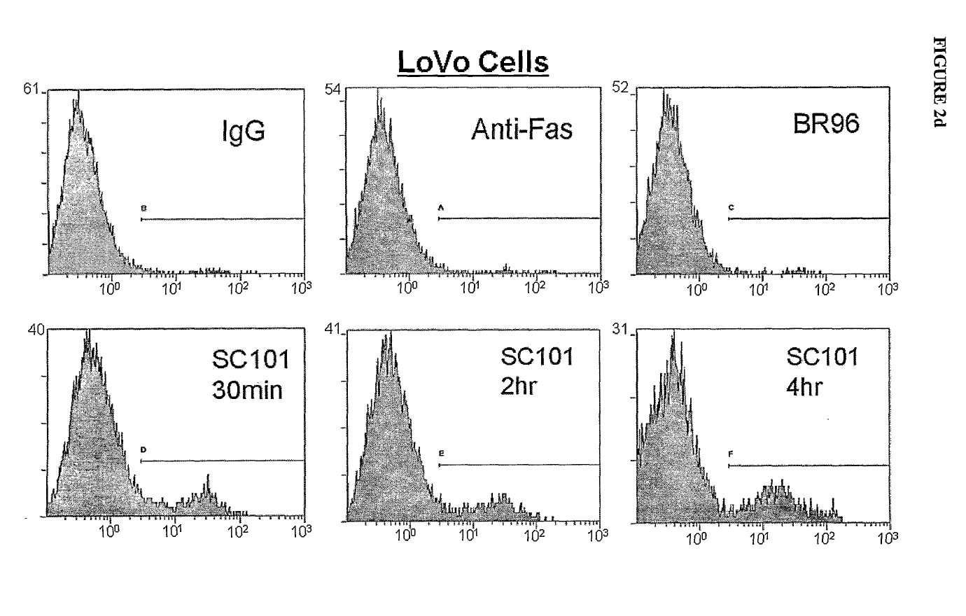

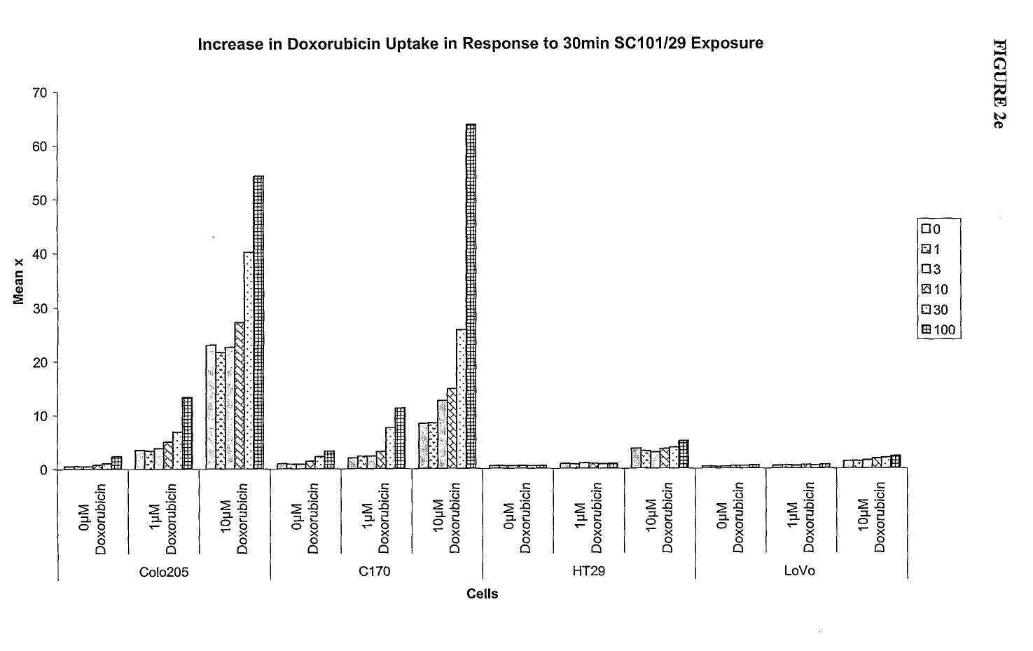

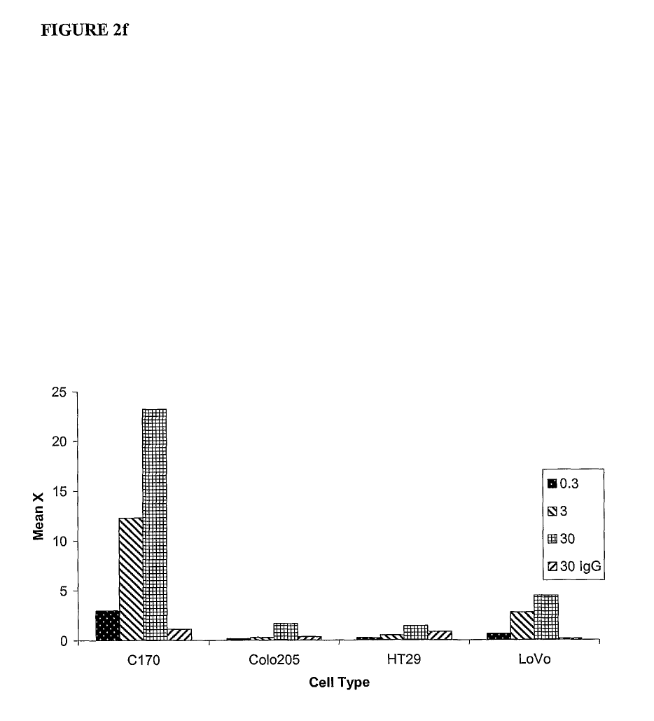

To assess the mechanism of cell death mediated by SC101/29 mAb, propidium iodide (PI), a fluorescent compound that under isotonic conditions can only enter non-viable cells, was used as a probe to identify cells that had lost their plasma membrane integrity. As shown in FIGS. 2a and 2b, SC101/29 mAb rapidly increased the permeability of Colo205 and C170 cells with more than 75% of cells staining strongly with PI as early as 30 min after treatment with the antibody. Under identical conditions fewer than 10% of cells took up PI when incubated for 4 hr with an anti-CD95 antibody known to induce apoptotic cell death or with control mouse IgG antibody. Similarly BR96, a mAb which only binds to Lewis^{y} and not to Lewis^{b}, also failed to show significant uptake of PI over a 4 hr period. Consistent with the inhibition of cell proliferation, SC101/29 mAb failed to induce PI uptake in HT-29 and LoVo cells which have low antigen expression.

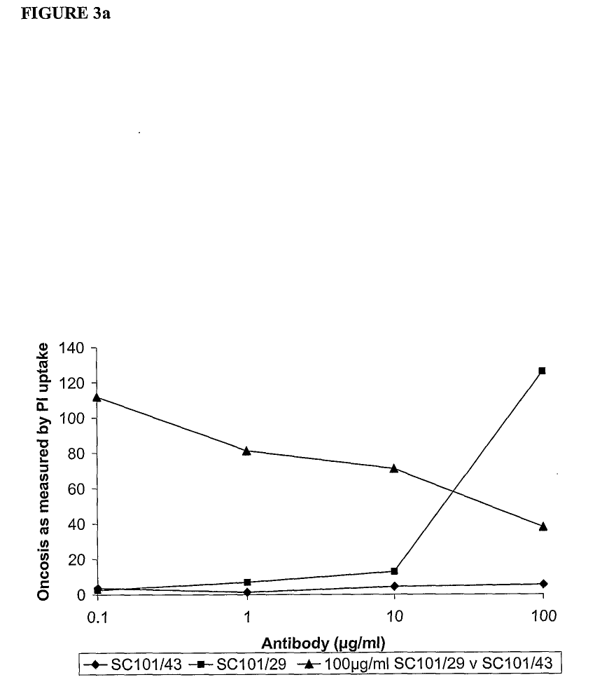

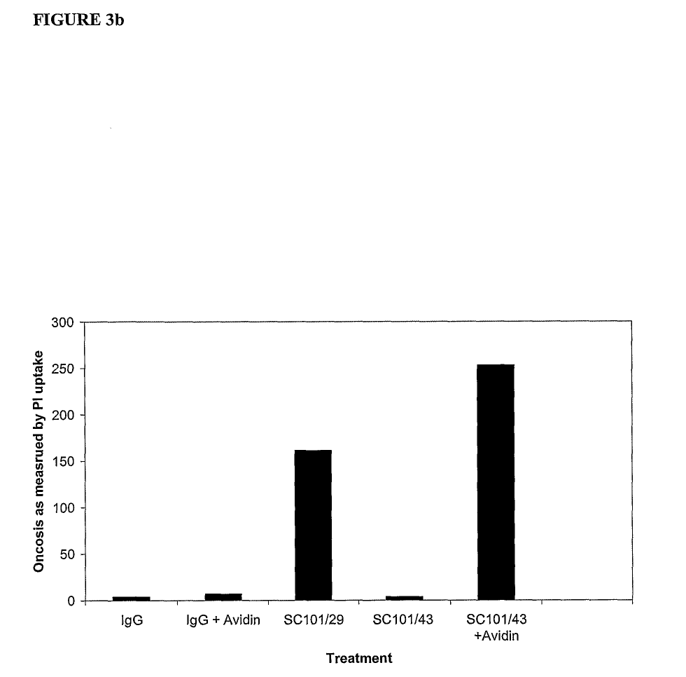

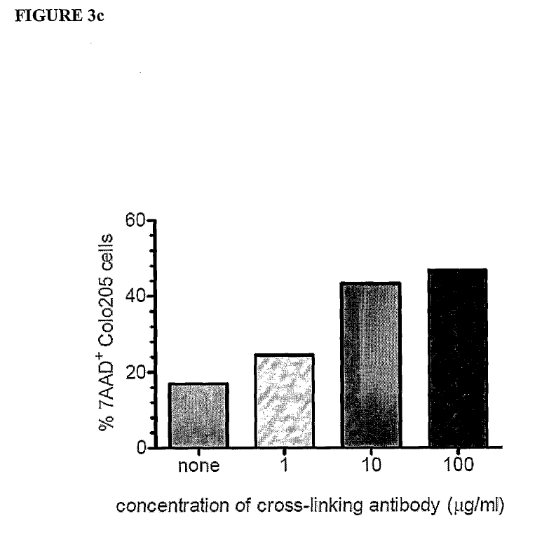

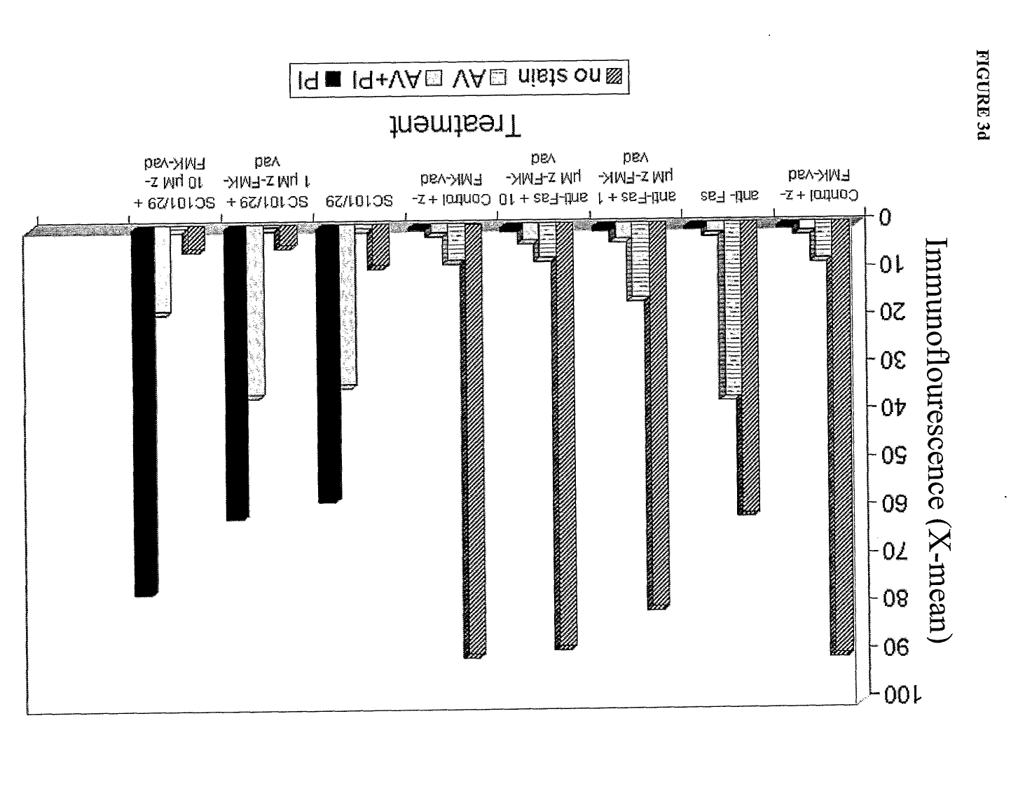

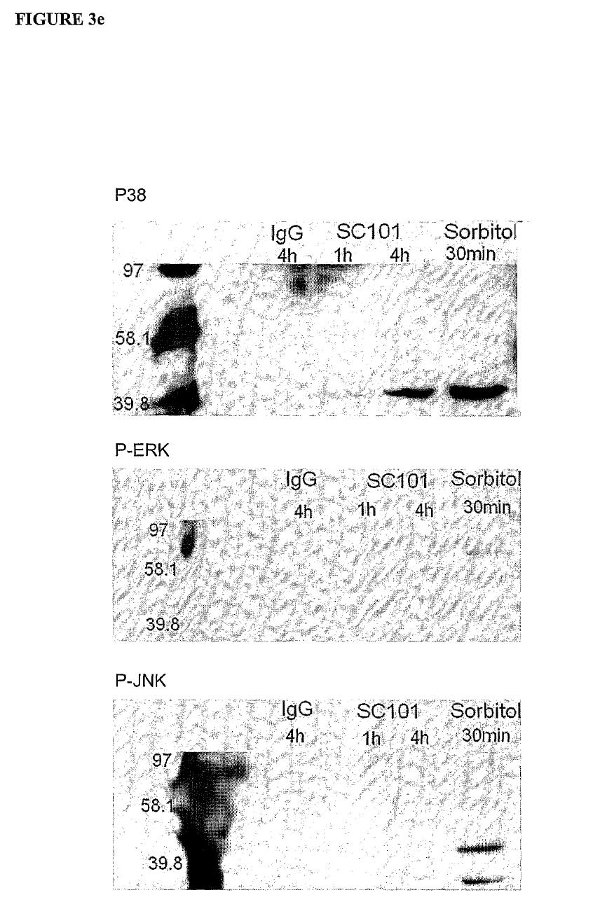

SC 101/43 was also analyzed for its ability to alter membrane permeability however in direct contrast to SC101/29 it failed to induce PI uptake (FIG. 3a). However, it was very effective at blocking the uptake of PI induced by SC 101/29 indicating that it binds at the same or a closely related epitope. If SC101/43 is cross-linked using avidin/biotin it can also induce efficient cell killing. Likewise cross-linking of a chimeric SC101/29 IgG₂ antibody leads to direct killing of colon tumour cells (FIG. 3c). As the SC101/29 is a mouse IgG3 that exist as natural dimers these results suggest that cross-linking of Lewis^{y} and Lewis^{b} is important for the oncosis which SC101/29 does naturally due to its dimeric structure, whereas SC101/43 being an IgG1 requires artificial cross-linking.

Drug Uptake in MDR Cell Lines

The loss in membrane integrity allows for access of small molecules into cells. This is illustrated with the fluorescent drug doxorubicin; following exposure of a range of cells to SC101/29 mAb, doxorubicin uptake was enhanced in cell lines expressing high levels of antigen and showing loss of membrane integrity (Colo205 and C170) but not in the cells with low antigen density (HT-29 and LoVo, FIG. 2e). This effect was even manifested in cells (C170) expressing p-glycoprotein (FIG. 2f) suggesting the membrane perturbation caused by SC101/29 inhibited excretion of the drug by this pump.

Inhibition of Caspase Dependent Cell Death Assay

Rapid loss of membrane integrity usually results in oncosis where the pores formed in the membrane continue to increase in size until they allow release of large cytoplasmic proteins, such as LDH. Whilst PI indicated a loss of integrity the concurrent release of LDH from C170 cells was not seen. The lack of LDH release queried the onset of oncosis in C170's, therefore, in order to ensure the PI staining identified could not be attributed to an artefact of classical apoptosis the pan caspase inhibitor, z-FMK-vad was utilised. In summary, whilst anti-Fas exposure had previously been seen to poorly induce apoptosis in C170 cells it could be seen that the inhibitor could completely reverse the effect of the antibody (FIG. 3d). Exposure of the tumour cells to SC101/29 mAb in the presence of z-FMK-vad had no discernable effect suggesting that the mode of death is independent of the classical apoptotic pathway.

Signalling Via Stress Related Kinases

Membrane permeability has been strongly associated with activation of p38 which phosphorylates HSP27 resulting in actin polymerisation. Weak p38 activation was observed in C170 cells after 1 hr with stronger activation after 4 hr (FIG. 3e). In contrast there was no activation of JNK or ERK. The stress activated protein kinase, p38 can also be induced by a range of chemotherapeutic agents including 5-FU. Therefore, to determine if SC101/29 could induce cell death in vivo and if this could synergise with 5-FU it was administered to mice transplanted with human xenografts.

In Vivo Studies

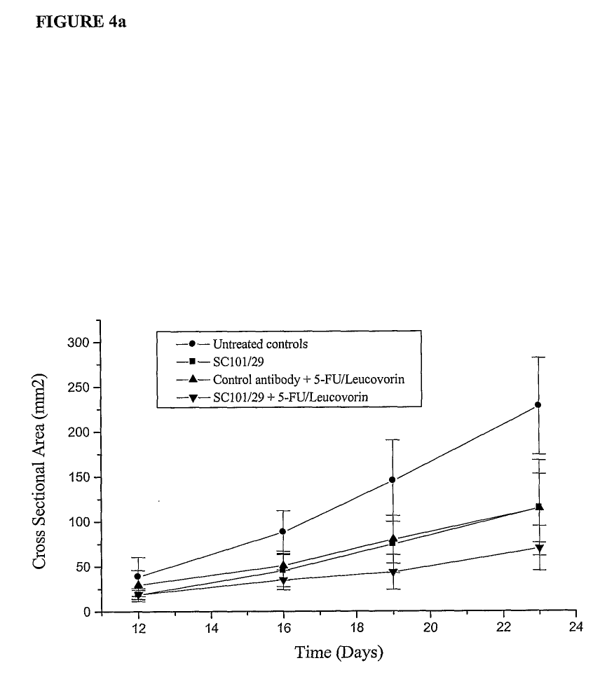

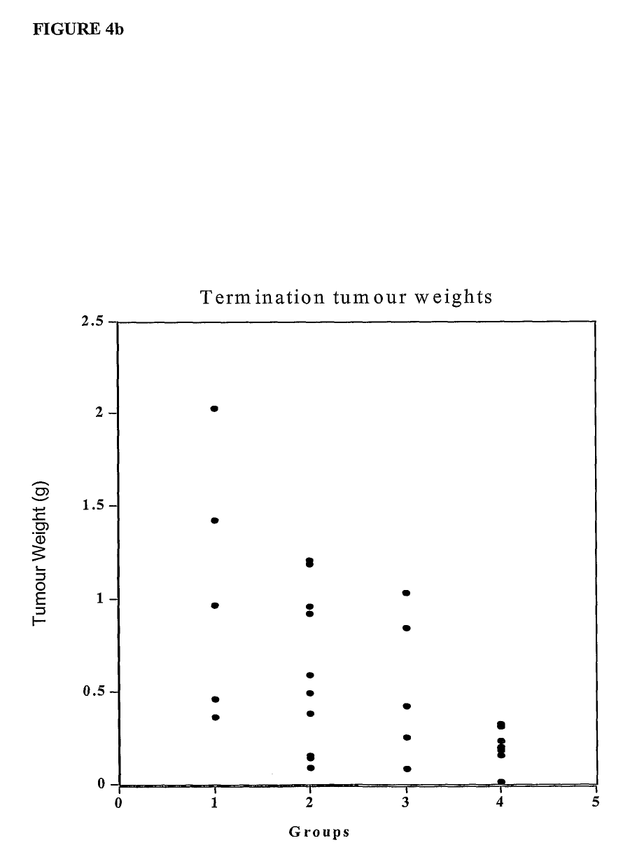

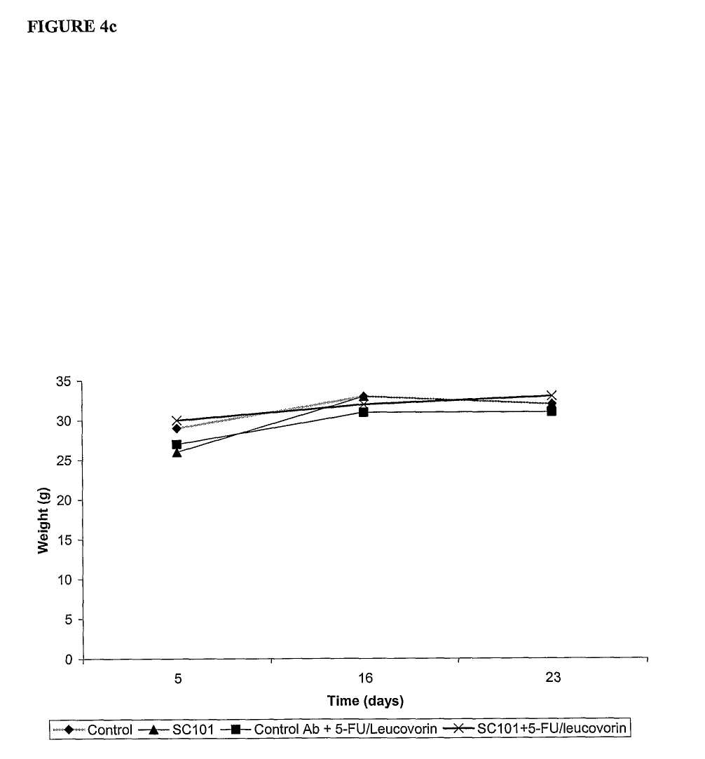

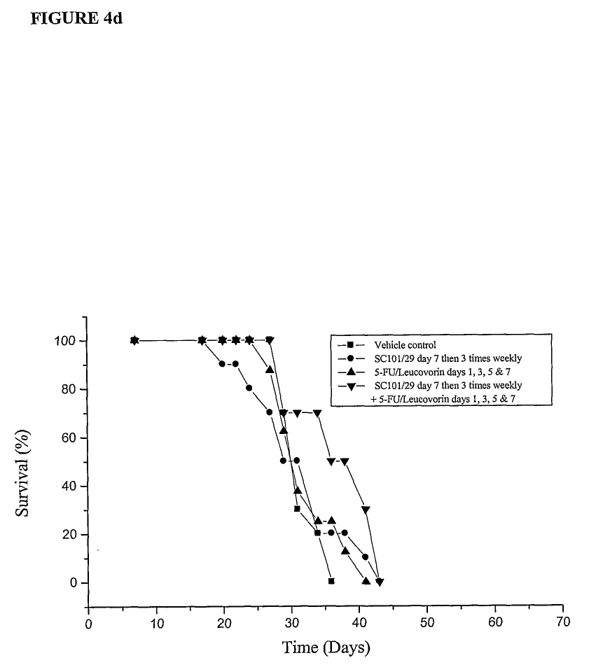

The antibody was administered 3 times weekly to mice either transplanted with 3 mm² extracts of C170 tumours, or to C170 tumours that had been allowed to grow for 5 days prior to administration of the antibody. Animals were treated either with SC101/29 mAb alone, 5-FU/leucovorin or a combination of both for 3 weeks. FIG. 4a shows that both SC101/29 mAb and 5-FU/leucovorin alone resulted in inhibition of tumour growth of freshly explanted tumours (p<0.004 ANOVA). In contrast, the combination of both showed additive inhibition of growth with only 2/10 mice showing any growth above the 0.3 g weight of the implanted tumours (FIG. 4b, p<0.02, ANOVA). This dose of SC101/29 mAb was well tolerated with all mice showing no loss of weight or any other gross pathology (FIG. 4c). When SC101/29 mAb was administered therapeutically to mice expressing C170 tumours in combination with 25 mg/kg 5-FU/leucovorin it significantly inhibited tumour growth and enhanced survival (FIG. 4d: p<0.0163 Log Rank).

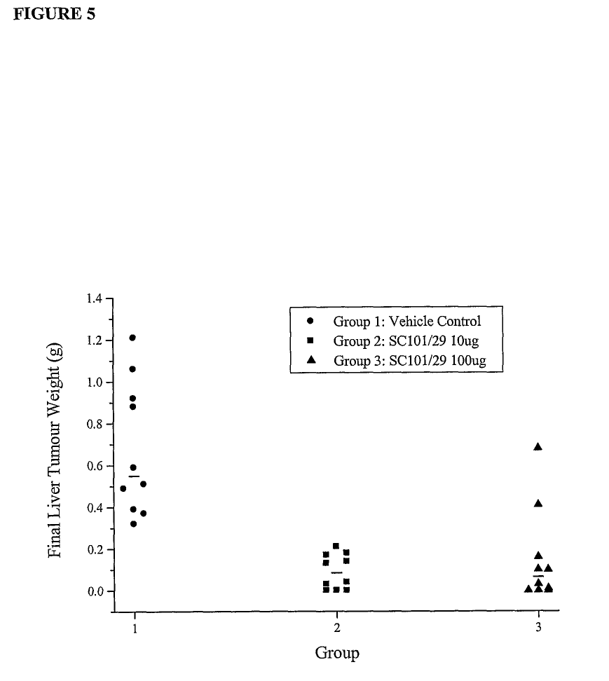

As the mechanism of cell killing suggested that binding of SC101/29 to micrometastatic cells should be very effective the therapeutic efficacy of SC101/29 mAb in preventing liver metastases was evaluated using the C170HM2 colorectal model. Mice were treated with the lower doses of 100 μg and 10 μg of SC101/29 mAb three times weekly for 6 weeks. At the termination of the study the weights of the excised C170HM2 liver tumour were measured. The median for each group was calculated and the data graphed as shown in FIG. 5. The median and mean values for each group were analysed for statistical significance by Mann-Whitney Test. The median values for 10 μg and 100 μg SC101/29 mAb (Groups 2 and 3) were found to be significant (p<0.001) from vehicle control (Group 1). As SC101/29 was so effective in this model even at low doses 5-FU was not required.

### EXAMPLE 3

Discussion

A range of Lewis^{y} antibodies have been identified but they consistently cross-react with Lewis^{x} and H-type 2 structures. This can lead to undesirable cross-reactivates with normal tissues leading to subsequent toxicities in clinical trials. For example, in a phase I study of murine mAb BR55-2, which cross-reacts with H antigen, there was haematuria in 6/12 patients and diahorea in 2/9 patients with only transient reductions in skin lesions seen in 3 patients (Tolcher et al. (1999) J. Clin. Oncol. 17:478-84). The LMB-1 immunotoxin (B3 antibody linked to pseudomonas endotoxin) directed against Lewis^{y} only resulted in responses in 5/38 patients (Pai et al. (1996) Nat. Med. 2:350-3). BR96 mAb cross-reacts with B blood group but not Lewis^{b}; gastrointestinal binding was dose limiting and shown to be related to antibody binding (Saleh et al. (2000) J. Clin. Oncol. 18:2282-92). Finally, a Lewis^{y} specific humanised antibody 3S193 has shown superior selectivity in binding studies (Scott et al. (2000) Cancer Research 60:3254-61) and has entered clinical trials.

The SC101 antibodies recognise a unique facet of both Lewis^{y} and Lewis^{b}. It may have been predicted that the SC101 antibodies would show strong cross-reactivity with a range of normal tissues expressing either Lewis^{y} or Lewis^{b} however, in contrast to other Lewis antibodies, only weak staining of mucin within the gastrointestinal tract was observed (Brown et al. (1983) Biosci. Rep. 3:163). This suggests that the gastrointestinal toxicity observed with BR96 mAb may be avoided with SC101 antibodies.

The SC101 antibodies induce tumour cell death in vitro without immune effectors cells and probably also in vivo as IgG3 antibodies are very poor mediators of CDC and ADCC. Of great interest was that the tumour cells needed to over-express Lewis^{y} and Lewis^{b} to be killed. At this level of expression 80% of gastrointestinal and 30% of ovarian/breast tumours would be susceptible to cell killing but normal tissues would be excluded providing a good therapeutic window. The mechanism of cell killing was very interesting as SC101/29 mAb caused rapid loss of membrane integrity. This is similar to cell death by oncosis which is observed in osmotically shocked and hyperoxia cells (Garmyn et al. (2001) J. Invest. Dermatol. 117:1290-5; Moriguchi et al. (1996) J. Biol. Chem. 271:26981-8; Romanshko et al. (2003) Free Radic. Biol. Med. 35:978-93; Shen et al. (2002) J. Biol. Chem. 277:45776-84; Tilley et al. (1996) FEBS Letters 395:133-6). However, traditionally oncosis progression then yields the release of cytosolic contents such as LDH (Chen et al. (2001) Toxicol. Appl. Pharmacol. 171:1-11). The mechanism of cell death seems to be related to the Lewis^{y}/Lewis^{b} structure, as BR96 a well characterised anti-Lewis^{y} antibody, failed to show any effect on plasma membranes. Lewis^{y} and Lewis^{b} also needed to be cross-linked, either by natural mouse IgG3 dimers or by avidin/biotin cross-linking of the IgG1 variants, to cause its membrane perturbation. Several other antibodies have been described that can induce oncotic like tumour cell death. These include an anti-porimin antibody that recognises a widely expressed glycoprotein (Ma et al. (2001) Proc. Natl. Acad. Sci. 98:9778-83), RE2 which recognises a renal associated glycoprotein (Matsuoka (1995) J. Exp. Med. 181:2007-2015) and RAV12 which recognises a novel glycotope. However, SC101 is unique as none of these antibodies have been shown to increase uptake of chemotherapeutic agents particularly in MDR cell lines.

PI uptake in cells was accompanied by p38 activation. This could explain the membrane permeability as p38 activation has recently been linked to phosphorylation of HSP27 which results in changes in microfilament dynamics (Deschesnes (2001) Mol. Biol. Cell 12:1569-82; Huot et al. (1998) J. Cell Biology 143:1361-73). This loss of membrane integrity is a rapid event and is upstream of cell death. Activation of p38 can result in caspase dependent or independent cell death (Deschesnes (2001) Mol. Biol. Cell 12:1569-82). As SC101/29 mAb failed to induce DNA fragmentation (data not shown) and inhibition of caspases using a pan caspase inhibitor z-FMK-vad did not appear to prevent cell death this would imply that the antibody is inducing caspase independent cell death. Colorectal tumours exhibit>70% mutations in the genes involved in the apoptotic response, any treatment which circumnavigates this response will be of considerable therapeutic value (Vogelstein (1988) N. Engl. J. Med. 319:525-32; Fulda et al. (2004) Curr. Cancer Drug Targets 4:569-76). One caspase independent mechanism involves the release of apoptosis inducing factor (AIF) from the mitochondria and its translocation to the nucleus where it contributes in an unknown manner to trigger nuclear condensation (Susin et al. (2000) J. Exp. Med. 192:571-80).

The stress related kinase p38 can also be induced by a range of chemotherapeutic agents and has been proposed to play a pivotal role in drug synergies (Olson & Hallahan (2004) Trends Mol Med 10:125-9). Lewis^{y} and Lewis^{b} is expressed on tumour glycoproteins and glycolipids and these can be upregulated in response to cellular stress. Indeed, upregulation of Lewis^{y} in response to chemotherapeutic stress (Flieger et al. (2001) Clin. Exp. Immunol. 123:9-14) and in particular to 5-FU has been previously reported. Therefore, as 5-FU/leucovrin is a standard chemotherapy for colorectal cancer, it can upregulate Lewis^{y} and activate p38 (Feng et al. (2002) Cancer Research 62:1920-6) the combination of SC101/29 and 5-FU/leucovorin were screened in vivo for their ability to inhibit growth of colon xenografts in nude mice. SC101/29 mAb alone and in combination with 5-FU/leucovorin significantly inhibited growth of both small and established tumours. If phase I clinical trials with a chimeric version of SC101/29 mAb show it to be safe, subsequent trials of the combination of 5-FU/leucovorin and SC101/29 mAb in comparison to drug alone in colorectal cancer patients following surgery would be initiated. This combination may be more effective than Panorex/5-FU as this antibody had no direct killing activity and relied on CDC and ADCC, which have limited success with solid tumours due to over-expression of complement regulatory molecules (Li et al. (2001) Br. J. Cancer 84:80-6).

The present inventors have shown that SC101/29 induces membrane permeability allowing up-take of small molecular weight (<800D) drugs but not release of large intracellular components such as LDH. SC101/29 also induces phosphorylation of the stress related kinase p38 but not JNK and finally induces caspase independent cell death. This is of particular interest as it has been shown that ceramide induces neuronal cell death through activation of p38 and release of multiple mitochrondrial proteins including AIF (Ghatan et al. (2000) J. Cell Biol. 150:335-47; Stoica et al. (2005) Mol. Cell Neurosci. 29:355-71). As Lewis^{y}/Lewis^{b} is predominantly expressed on a glycolipid its degradation may result in an increase in ceramide and subsequent activation of p38. As SC101/29 does not induce cell death by classically described pathways it may be particularly effective in tumour cells that have evolved mechanism to make them resistant to classical apoptosis or oncosis. However the pathway needs more careful elucidation to allow further validation of our killing assays to assess potential mechanism of resistance and to allow us to identify the optimal tumour targets.

In conclusion, SC101/29 is a novel mAb that recognises Lewis^{y} and Lewis^{b} and directly induces cell death by inducing loss of membrane integrity. This is the first description of an antibody recognising a blood group antigen that can selectively kill tumour cells in an oncolytic manner. The role of p38 in therapeutic synergy reactions is becoming increasingly evident. SC101/29 shows enhanced killing in combination with 5-FU/leucovorin and could be used in combination with these drugs to reduce toxicity and increase efficacy of treatment for cancer.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A binding complex comprising antibodies or antigen-binding fragments which bind to both Lewis^{y} and Lewis^{b} antigens, wherein the antibodies or antigen-binding fragments are in the form of multimers, and wherein the antibodies or antigen-binding fragments do not naturally form multimers.

2. The binding complex according to claim 1 wherein the multimer is a homo- or hetero-multimer.

3. The binding complex according to claim 1 wherein the multimer is a homo- or hetero-dimer.

4. The binding complex according to claim 1 wherein the multimer comprises an IgG antibody.

5. The binding complex according to claim 1 wherein the multimer is a hetero-dimeric IgG₂/IgG₃ antibody.

6. The binding complex according to claim 1 wherein the multimer comprises antibodies or antigen-binding fragments which are not derived from the same species.

7. The binding complex according to claim 1 wherein the multimer is a dimeric IgG antibody.

8. The binding complex according to claim 1 wherein the antigen-binding fragments are selected from the group consisting of multibodies, domain antibodies, Fv fragments, Fd fragments, Fab fragments, F(ab′)₂ fragments, complementarity determining regions (CDRs) and CDRs fused through a cognate framework region.

9. The binding complex according to claim 8 wherein the multibodies are diabodies or triabodies.

10. The binding complex according to claim 9 wherein the multibodies are diabodies.

11. The binding complex according to claim 8 wherein the multibodies further comprise a modified Fc domain.

12. A method for inducing cell death in tumour cells over-expressing Lewis^{y} and Lewis^{b} antigens, comprising:
administering to a subject an effective amount of a binding complex selected from the group consisting of an antibody and an antigen-binding fragment which binding complex binds to both Lewis^{y} and Lewis^{b} antigens, wherein the binding complex forms multimers, which multimers do not occur in nature.

13. A method for treating cancer in a subject, comprising:
administering to a subject an effective amount of a binding complex selected from the group consisting of an antibody and an antigen-binding fragment which binding complex binds to both Lewis^{y} and Lewis^{b} antigens, wherein the binding complex forms multimers, which multimers do not occur in nature.

14. The method according to claim 13 wherein the cancer is selected from the group consisting of breast cancer, lung cancer, colon cancer, ovarian cancer, prostate cancer and pancreatic cancer.

15. The method according to claim 12 wherein the binding complex is formulated as a pharmaceutical composition for administration to the subject.

16. The method according to claim 12 wherein the binding complex is administered as an injectable solution.

17. The method according to claim 12 wherein the binding complex is administered intravenously.

18. The method according to claim 12 wherein the binding complex is administered to a human.

19. A pharmaceutical composition for inducing cell death in tumour cells over-expressing Lewis^{y} and Lewis^{b} antigens, the composition comprising a therapeutically effective amount of a binding complex comprising antibodies or antigen-binding fragments which bind to both Lewis^{y} and Lewis^{b} antigens, together with a pharmaceutically acceptable carrier, wherein the antibodies or antigen-binding fragments are in the form of multimers, and wherein the antibodies or antigen-binding fragments do not naturally form multimers.

20. (canceled)

21. (canceled)

22. The method according to claim 13 wherein the binding complex is formulated as a pharmaceutical composition for administration to the subject.

23. The method according to claim 13 wherein the binding complex is administered as an injectable solution.

24. The method according to claim 13 wherein the binding complex is administered intravenously.

25. The method according to claim 13 wherein the binding complex is administered to a human.
